(19) United States
(12) Patent Application Publication
(10) Pub. No.: US 2021/0340240 A1
(43) Pub. Date: Nov. 4, 2021

(54) COMBINATION OF A BIG-H3 ANTAGONIST AND AN IMMUNE CHECKPOINT INHIBITOR FOR THE TREATMENT OF SOLID TUMOR

(71) Applicants: INSERM (Institut National de la Santé et de la Recherche Médicale, Paris (FR); Centre National de la Recherche Scientifique (CNRS), Paris (FR); Université Claude Bernard - Lyon 1, Villeurbanne (FR); Centre Leon Berard, Lyon (FR); KIST (Korea Institute of Science and Technology), Seoul (KR)

(72) Inventor: Ana HENNINO, Lyon (FR)

(21) Appl. No.: 17/283,607

(22) PCT Filed: Oct. 17, 2019
(86) PCT No.: PCT/EP2019/078246

§ 371 Date: Apr. 8, 2021

(30) Foreign Application Priority Data
Oct. 18, 2018 (EP) 18306370.0

(51) Int. Cl. C07K 16/22; C07K 16/28; A61P 35/04

(52) U.S. Cl. CPC C07K 16/22; C07K 16/2818; A61K 2039/507; A61P 35/04; C07K 16/2827

(57) ABSTRACT

To study the mechanism of βig-h3 modulation of the anti-tumoral immune response in pancreatic cancer, Inventors took advantage of engineered mouse models of spontaneous pancreatic neoplasia and cancer to evaluate the effect of depleting βig-h3 on the modulation of anti-tumor immunity and its subsequent impact on tumour growth alone and in combination with an immune checkpoint inhibitor. This association proved to be effective in vivo in this model showing a synergic effect of the therapeutic combination. Accordingly, the present invention relates to a combination of (i) an immune checkpoint inhibitor, and (ii) a βig-h3 antagonist, for simultaneous or sequential use in the treatment of a patient suffering from solid tumor, e.g. a pancreatic cancer. The present invention also provides a βig-h3 antagonist, for use in a method for enhancing sensitivity of a patient suffering from a solid tumor to an immune checkpoint inhibitor.

### FIELD OF THE INVENTION

The present invention relates to a combination of (i) a βig-h3 antagonist, and (ii) an immune checkpoint inhibitor, for the simultaneous or sequential use in the treatment of a patient suffering from a solid tumor, e.g. a pancreatic cancer. The present invention also provides a βig-h3 antagonist, for use in a method for enhancing sensitivity of a patient suffering from tumor to an immune checkpoint inhibitor.

### BACKGROUND OF THE INVENTION

Pancreatic ductal adenocarcinoma (PDA) is a highly aggressive cancer with a median survival of less than 6 months and a 5-year survival rate of 3-5%¹. PDA evolves through a series of pancreatic intraepithelial neoplasias (PanINs) that are accompanied by genetic modifications. Of these, the earliest and most ubiquitous is the oncogenic activation of Kras². In addition to the molecular and histological alterations that define cancer cells, a hallmark of PDA is the prominent stromal reaction that surrounds the neoplastic cells. The cellular component of the stroma includes immune cells, such as lymphocytes, macrophages and myeloid-derived suppressor cells (MDSCs), along with vascular and neural elements (i.e., endothelial cells and neurons, respectively) as well as cancer-associated fibroblasts (CAFs).

It is now well-established that activated pancreatic stellate cells (PSCs) are the major population of cells that is responsible for the production of this collagenous stroma³. PSCs represent, at steady state, approximately 4% of the pancreas. They become activated upon inflammation and are then converted into CAFs. Recent studies have demonstrated that CAFs are able to attract and sequester CD8+ T cells in the extra-tumoral compartment. This effect dampens their contact with and consequent clearing of tumor cells⁴. Several studies performed in mice have shown that depleting CAFs abolishes immune suppression,^{5, 6} indicating that they play an important role in modulating the local anti-tumoral response. In most solid tumors, as in PDA, CD8+ T cell infiltration into the tumor is a factor associated with a good prognosis^{7, 8}. PDA patients with high densities of CD8+ T cells in the juxtatumoral compartment have longer survival times than patients with lower densities^{4, 9}. Therefore, restoring the anti-tumoral CD8+ T cell response might be very important in PDA.

Immune checkpoint blockade has elicited clinical responses in some patients with different advanced malignancies (ie melanoma) but has not been effective in PDAC, suggesting that other factors including mechanical tension generated in desmoplastic tumor microenvironment may limit T cell activity¹⁰. The immune cells do not penetrate the parenchyma of these tumours but instead are retained in the stroma that surrounds nests of tumour cells^{11, 12} After treatment with anti-PD-L1/PD-1 agents, stroma-associated T cells can show evidence of activation and proliferation but not infiltration associated with no clinical responses¹⁰.

βig-h3 (also known as TGFβi) is a 68-kDa ECM protein that was first isolated from A549 human lung adenocarcinoma cells that were treated with TGF-β¹³. The physiological functions of βig-h3 have been proposed to include cell-matrix interactions and cell migration¹⁴. βig-h3 has also been shown to bind to several ECM molecules, such as collagens I, II, and IV and fibronectin, proteoglycans and periostin^{15, 16}. At the cell surface, βig-h3 has been shown to interact with various integrins, including α_{V}β₃,^{17, 18} α₁β₁¹⁸ and α_{V}β₅¹⁹. It was recently showed that βig-h3 repressed diabetogenic T-cell activation by interfering with early factors in the TCR signaling pathway, such as Lck²⁰. Inventors previously found that βig-h3 expression was increased in some cancers, including pancreatic cancer²¹, whereas in other cancers, such as ovarian cancer and multiple myeloma, the levels of βig-h3 were reduced^{22, 23}. Because the expression of βig-h3 was higher in pancreatic cancer, which is associated with an increase in immune suppression, Inventors demonstrated that βig-h3 play a role in directly modulating the anti-tumoral immune response by blocking inhibiting CD8+ T cell activation (see WO2017/158043).

To conclude, Immune checkpoint blockade have been tested as anticancer therapy but have not proven liable to completely treat all individuals afflicted with cancer, and especially solid tumors such as pancreatic cancers, which are notably associated with poor prognosis. Thus, there is a need for new therapeutic alternatives that could provide new perspectives in particular in pancreatic cancer treatment.

### SUMMARY OF THE INVENTION

The present invention relates to a combination of a βig-h3 antagonist and of an immune checkpoint inhibitor for simultaneous or sequential use in the treatment of a patient suffering from solid tumor, and in particularly from pancreatic cancer. The present invention also provides a βig-h3 antagonist, for use in a method for enhancing sensitivity of a patient suffering from solid tumor to an immune checkpoint inhibitor.

### DETAILED DESCRIPTION OF THE INVENTION

The present invention arises from the unexpected finding by the inventors that a βig-h3 antagonist, such as a neutralizing βig-h3 antibodies, acts synergistically with an immune checkpoint inhibitor (antibody anti PD1), to promote cancer cell apoptosis and prevent tumour growth.

To study the mechanism of βig-h3 modulation of the anti-tumoral immune response in pancreatic cancer, Inventors took advantage of engineered mouse models of spontaneous pancreatic neoplasia and cancer that were based on KrasG12D activation in pancreatic cells^{24, 25}. Using these models, they evaluated the effect of depleting βig-h3 on the modulation of anti-tumor immunity and its subsequent impact on tumour growth alone and in combination with an immune checkpoint inhibitor (see FIGS. 1 and 2). This association proved to be effective in vivo in these model showing a synergic effect of the therapeutic combination.

Without bound to any theory, inventors demonstrate that CAF-secreted βig-h3 play an important role in the stiffening observed in tumor microenvironment (see FIGS. 3 and 4) and that the depletion of this protein have an impact on the immunosuppression but also may have a role on the mechanical release of the stroma of the anti-tumoral CD8+ T cell.

Accordingly, the present inventors demonstrate the effect of neutralizing a newly identified stromal target (βig-h3) in respect to the mechanical tension release and penetration of anti-tumor T cells (FIG. 3). Accordingly the benefit of using anti-stromal therapy in order to enhance response to anti-PD-1 check point immunotherapy was well established and allows potential for combined immune and specific stromal therapy for solid tumor such as pancreatic cancer.

Combination of a βIg-h3 Antagonist with an Immune Checkpoint Inhibitor, for Use in the Treatment of Solid Tumor

Therefore, the present invention provides a combination of

i. a βig-h3 antagonist; and

ii. an immune checkpoint inhibitor;

for simultaneous or sequential use in the treatment of a solid tumor.

The present invention also provides a βig-h3 antagonist, for use in a method for enhancing sensitivity of a patient suffering from solid tumor to an immune checkpoint inhibitor.

In its broadest meaning, the term “treating” or “treatment” refers to reversing, alleviating, inhibiting the progress of the disorder or condition to which such a term applies, or one or more symptoms of such a disorder or condition.

An “βig-h3 antagonist” refers to a molecule (natural or synthetic) capable of neutralizing, blocking, inhibiting, abrogating, reducing or interfering with the activities of βig-h3 including, for example, reduction or blocking the interaction between βig-h3 and αVβ3 integrin and/or reduction or blocking the interaction between βig-h3 and collagen. βig-h3 antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include, antagonist variants of the protein, siRNA molecules directed to a protein, antisense molecules directed to a protein, aptamers, and ribozymes against a protein. For instance, the βig-h3 antagonist may be a molecule that binds to βig-h3 and neutralizes, blocks, inhibits, abrogates, reduces or interferes with the biological activity of βig-h3 (such as blocking the anti-tumoral immune response). More particularly, the βig-h3 antagonist according to the invention is an anti-βig-h3 antibody.

By “biological activity” of a βig-h3 is meant inhibiting CD8+ T cell activation (blocking the anti-tumoral immune response) and inducing stiffening of tumor microenvironment (TME or tumoral stroma).

Tests for determining the capacity of a compound to be βig-h3 antagonist are well known to the person skilled in the art. In a preferred embodiment, the antagonist specifically binds to βig-h3 in a sufficient manner to inhibit the biological activity of βig-h3. Binding to βig-h3 and inhibition of the biological activity of βig-h3 may be determined by any competing assays well known in the art. For example, the assay may consist in determining the ability of the agent to be tested as βig-h3 antagonist to bind to βig-h3. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an antagonist that “specifically binds to βig-h3” is intended to refer to an inhibitor that binds to human βig-h3 polypeptide with a KD of 1 μM or less, 100 nM or less, 10 nM or less, or 3 nM or less. Then a competitive assay may be settled to determine the ability of the agent to inhibit biological activity of βig-h3. The functional assays may be envisaged such evaluating the ability to inhibit a) induction of stiffening of TME and/or b) inhibition of CD8+ T cell activation (see example/method related with Functional T cell Suppression Assay).

The skilled in the art can easily determine whether a βig-h3 antagonist neutralizes, blocks, inhibits, abrogates, reduces or interferes with a biological activity of βig-h3. To check whether the βig-h3 antagonist binds to βig-h3 and/or is able to inhibit stiffening of TME and/or blocking the inhibiting CD8+ T cell activation in the same way than the initially characterized blocking βig-h3 antibody and/or binding assay and/or a collagen I thick fiber assay and/or or a inhibiting CD8+ T cell activation assay may be performed with each antagonist. For instance inhibiting CD8+ T cell activation can be assessed by detecting cells expressing activation markers with antibody anti-CD69 and anti-CD44 (CD8+ T cells) as described in Patry and al,²⁰ (or see the Functional T cell Suppression Assay in example method) and collagen I thick fiber assay can be measured by atomic force microscopy or polarized light after Sirius Red staining (see example section).

Accordingly, the βig-h3 antagonist may be a molecule that binds to βig-h3 selected from the group consisting of antibodies, aptamers, and polypeptides.

The skilled in the art can easily determine whether a βig-h3 antagonist neutralizes, blocks, inhibits, abrogates, reduces or interferes with a biological activity of βig-h3: (i) binding to βig-h3 and/or (ii) inducing stiffening of TME and/or (iii) inhibiting CD8+ T cell activation.

Accordingly, in a specific embodiment the βig-h3 antagonist directly binding to βig-h3 and inhibits the inhibition of CD8+ T cell activation (or restore CD8+ T cell activation) and stiffening of TME.

As used herein, the expression “tumor microenvironment (TME)” or “tumoral stroma” (both expressions will be used interchangeably) has its general meaning in the art and refers to the cellular environment in which the tumor exists, including surrounding blood vessels, immune cells, fibroblasts, bone marrow-derived inflammatory cells, lymphocytes, signalling molecules and the extracellular matrix (ECM) (Joyce, J A.; et al. (April 2015). Science Magazine. pp. 74-80; Spill, F.; et al. Current Opinion in Biotechnology. 40: 41-48)). The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of cancerous cells (Korneev, K V; et al (January 2017). “Cytokine. 89: 127-135.).

As used herein, the expression “immune checkpoint inhibitor” or “checkpoint blockade cancer immunotherapy agent” (both expressions will be used interchangeably) has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. In particular, the immune checkpoint inhibitor of the present invention is administered for enhancing the proliferation, migration, persistence and/or cytotoxic activity of CD8+ T cells in the subject and in particular the tumor-infiltrating of CD8+ T cells of the subject. As used herein “CD8+ T cells” has its general meaning in the art and refers to a subset of T cells that express CD8 on their surface. They are MHC class I-restricted, and function as cytotoxic T cells. “CD8+ T cells” are also called CD8+ T cells are called cytotoxic T lymphocytes (CTL), T-killer cell, cytolytic T cells, CD8+ T cells or killer T cells. CD8 antigens are members of the immunoglobulin supergene family and are associative recognition elements in major histocompatibility complex class I-restricted interactions. The ability of the immune checkpoint inhibitor to enhance T CD8 cell killing activity may be determined by any assay well known in the art. Typically said assay is an in vitro assay wherein CD8+ T cells are brought into contact with target cells (e.g. target cells that are recognized and/or lysed by CD8+ T cells). For example, the immune checkpoint inhibitor of the present invention can be selected for the ability to increase specific lysis by CD8+ T cells by more than about 20%, preferably with at least about 30%, at least about 40%, at least about 50%, or more of the specific lysis obtained at the same effector: target cell ratio with CD8+ T cells or CD8 T cell lines that are contacted by the immune checkpoint inhibitor of the present invention, Examples of protocols for classical cytotoxicity assays are conventional.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211).

Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834).

Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057. In some embodiments, the immune checkpoint inhibitor is an Indoleamine 2,3-dioxygenase (IDO) inhibitor, preferably an IDO1 inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is an anti-TIGIT (T cell immunoglobin and ITIM domain) antibody.

In a preferred embodiment, the checkpoint blockade cancer immunotherapy agent is a CTLA4 blocking antibody, such as Ipilimumab, or a PD-1 blocking antibody, such as Nivolumab or Pembrolizumab, or a combination thereof.
In a particular embodiment, the immune checkpoint inhibitor consist in the PD-1 blocking antibody (Pembrolizumab) comprising:
   a heavy chain having a sequence set forth as SEQ ID NO:_1
   a light chain having a sequence set forth as SEQ ID NO:_2

The sequences of Pembrolizumab antibody are indicated in the following Table 1:

| Mab | |
|---|---|
| Pembrolizumab | |
| domains | Sequence |
| VH: | QVQLVQSGVEVKKPGASVKVSCKASGYTFTNYYM |
| SEQ ID NO:_1 | YWVRQAPGQGLEWMGGINPSNGGTNFNEKFKNRV |
| | TLTTDSSTTTAYMELKSLQFDDTAVYYCARRDYR |
| | FDMGFDYWGQGTTVTVSSASTKGPSVFPLAPCSR |
| | STSESTAALGCLVKDYFPEPVTVSWNSGALTSGV |
| | HTFPAVLQSSGLYSLSSVVTVPSSSLGTKTYTCN |
| | VDHKPSNTKVDKRVESKYGPPCPPCPAPEFLGGP |
| | SVFLFPPKPKDTLMISRTPEVTCVVVDVSQEDPE |
| | VQFNWYVDGVEVHNAKTKPREEQFNSTYRVVSVL |
| | TVLHQDWLNGKEYKCKVSNKGLPSSIEKTISKAK |
| | GQPREPQVYTLPPSQEEMTKNQVSLTCLVKGFYP |
| | SDIAVEWESNGQPENNYKTTPPVLDSDGSFFLYS |
| | RLTVDKSRWQEGNVFSCSVMHEALHNHYTQKSLS |
| | LSLGK |
| VL: | EIVLTQSPATLSLSPGERATLSCRASKGVSTSGY |
| SEQ ID NO:_2 | SYLHWYQQKPGQAPRLLIYLASYLESGVPARFSG |
| | SGSGTDFTLTISSLEPEDFAVYYCQHSRDLPLTF |
| | GGGTKVEIKRTVAAPSVFIFPPSDEQLKSGTASV |
| | VCLLNNFYPREAKVQWKVDNALQSGNSQESVTEQ |
| | DSKDSTYSLSSTLTLSKADYEKHKVYACEVTHQG |
| | LSSPVTKSFNRGEC |

In a particular embodiment, the immune checkpoint inhibitor consist in the PD-1 blocking antibody (Nivolumab) comprising:
   a heavy chain having a sequence set forth as SEQ ID NO:_3
   a light chain having a sequence set forth as SEQ ID NO:_4

The sequences of Nivolumab antibody are indicated in the following Table 2:

| Mab | |
|---|---|
| Nivolumab | |
| domains | Sequence |
| VH: | QVQLVESGGGVVQPGRSLRLDCKASGITFSNSGM |
| SEQ ID NO:_3 | HWVRQAPGKGLEWVAVIWYDGSKRYYADSVKGRF |
| | TISRDNSKNTLFLQMNSLRAEDTAVYYCATNDDY |
| | WGQGTLVTVSSASTKGPSVFPLAPCSRSTSESTA |
| | ALGCLVKDYFPEPVTVSWNSGALTSGVHTFPAVL |
| | QSSGLYSLSSVVTVPSSSLGTKTYTCNVDHKPSN |
| | TKVDKRVESKYGPPCPPCPAPEFLGGPSVFLFPP |
| | KPKDTLMISRTPEVTCVVVDVSQEDPEVQFNWYV |
| | DGVEVHNAKTKPREEQFNSTYRVVSVLTVLHQDW |
| | LNGKEYKCKVSNKGLPSSIEKTISKAKGQPREPQ |
| | VYTLPPSQEEMTKNQVSLTCLVKGFYPSDIAVEW |
| | ESNGQPENNYKTTPPVLDSDGSFFLYSRLTVDKS |
| | RWQEGNVFSCSVMHEALHNHYTQKSLSLSLGK |
| VL: | EIVLTQSPATLSLSPGERATLSCRASQSVSSYLA |
| SEQ ID NO:_4 | WYQQKPGQAPRLLIYDASNRATGIPARFSGSGSG |
| | TDFTLTISSLEPEDFAVYYCQQSSNWPRTFGQGT |
| | KVEIKRTVAAPSVFIFPPSDEQLKSGTASVVCLL |
| | NNFYPREAKVQWKVDNALQSGNSQESVTEQDSKD |
| | STYSLSSTLTLSKADYEKHKVYACEVTHQGLSSP |
| | VTKSFNRGEC |

In a particular embodiment, the immune checkpoint inhibitor consist in the PD-1 blocking antibody (Atezolizumab) comprising:
   a heavy chain having a sequence set forth as SEQ ID NO:_5
   a light chain having a sequence set forth as SEQ ID NO:_6

The sequences of Atezolizumab antibody are indicated in the following Table 3:

| Mab | |
|---|---|
| Atezolizumab | |
| domains | Sequence |
| VH: | EVQLVESGGGLVQPGGSLRLSCAASGFTFSDSWIH |
| SEQ ID NO:_5 | WVRQAPGKGLEWVAWISPYGGSTYYADSVKGRFTI |
| | SADTSKNTAYLQMNSLRAEDTAVYYCARRHWPGGF |
| | DYWGQGTLVTVSSASTKGPSVFPLAPSSKSTSGGT |
| | AALGCLVKDYFPEPVTVSWNSGALTSGVHTFPAVL |
| | QSSGLYSLSSVVTVPSSSLGTQTYICNVNHKPSNT |
| | KVDKKVEPKSCDKTHTCPPCPAPELLGGPSVFLFP |
| | PKPKDTLMISRTPEVTCVVVDVSHEDPEVKFNWYV |
| | DGVEVHNAKTKPREEQYASTYRVVSVLTVLHQDWL |
| | NGKEYKCKVSNKALPAPIEKTISKAKGQPREPQVY |
| | TLPPSREEMTKNQVSLTCLVKGFYPSDIAVEWESN |
| | GQPENNYKTTPPVLDSDGSFFLYSKLTVDKSRWQQ |
| | GNVFSCSVMHEALHNHYTQKSLSLSPGK |
| VL: | DIQMTQSPSSLSASVGDRVTITCRASQDVSTAVAW |
| SEQ ID NO:_6 | YQQKPGKAPKLLIYSASFLYSGVPSRFSGSGSGTD |
| | FTLTISSLQPEDFATYYCQQYLYHPATFGQGTKVE |
| | IKRTVAAPSVFIFPPSDEQLKSGTASVVCLLNNFY |
| | PREAKVQWKVDNALQSGNSQESVTEQDSKDSTYSL |
| | SSTLTLSKADYEKHKVYACEVTHQGLSSPVTKSFN |
| | RGEC |

In a particular embodiment, the immune checkpoint inhibitor consist in the PD-1 blocking antibody (Avelumab) comprising:
   a heavy chain having a sequence set forth as SEQ ID NO:_7
   a light chain having a sequence set forth as SEQ ID NO:_8

The sequences of Avelumab antibody are indicated in the following Table 4:

| Mab | |
|---|---|
| Avelumab | |
| domains | Sequence |
| VH: | EVQLLESGGGLVQPGGSLRLSCAASGFTFSSYIM |
| SEQ ID NO:_7 | MWVRQAPGKGLEWVSSIYPSGGITFYADTVKGRF |
| | TISRDNSKNTLYLQMNSLRAEDTAVYYCARIKLG |
| | TVTTVDYWGQGTLVTVSSASTKGPSVFPLAPSSK |
| | STSGGTAALGCLVKDYFPEPVTVSWNSGALTSGV |
| | HTFPAVLQSSGLYSLSSVVTVPSSSLGTQTYICN |
| | VNHKPSNTKVDKKVEPKSCDKTHTCPPCPAPELL |
| | GGPSVFLFPPKPKDTLMISRTPEVTCVVVDVSHE |
| | DPEVKFNWYVDGVEVHNAKTKPREEQYNSTYRVV |
| | SVLTVLHQDWLNGKEYKCKVSNKALPAPIEKTIS |
| | KAKGQPREPQVYTLPPSRDELTKNQVSLTCLVKG |
| | FYPSDIAVEWESNGQPENNYKTTPPVLDSDGSFF |
| | LYSKLTVDKSRWQQGNVFSCSVMHEALHNHYTQK |
| | SLSLSPGK |
| VL: | QSALTQPASVSGSPGQSITISCTGTSSDVGGYNY |
| SEQ ID NO:_8 | VSWYQQHPGKAPKLMIYDVSNRPSGVSNRFSGSK |
| | SGNTASLTISGLQAEDEADYYCSSYTSSSTRVFG |
| | TGTKVTVLGQPKANPTVTLFPPSSEELQANKATL |
| | VCLISDFYPGAVTVAWKADGSPVKAGVETTKPSK |
| | QSNNKYAASSYLSLTPEQWKSHRSYSCQVTHEGS |
| | TVEKTVAPTECS |

In a particular embodiment, the immune checkpoint inhibitor consist in the PD-1 blocking antibody (Durvalumab) comprising:
   a heavy chain having a sequence set forth as SEQ ID NO:_9
   a light chain having a sequence set forth as SEQ ID NO:_10

The sequences of Durvalumab antibody are indicated in the following Table 5:

| Mab | |
|---|---|
| Durvalumab | |
| domains | Sequence |
| VH: | EVQLVESGGGLVQPGGSLRLSCAASGFTFSRYWM |
| SEQ ID NO:_9 | SWVRQAPGKGLEWVANIKQDGSEKYYVDSVKGRF |
| | TISRDNAKNSLYLQMNSLRAEDTAVYYCAREGGW |
| | FGELAFDYWGQGTLVTVSSASTKGPSVFPLAPSS |
| | KSTSGGTAALGCLVKDYFPEPVTVSWNSGALTSG |
| | VHTFPAVLQSSGLYSLSSVVTVPSSSLGTQTYIC |
| | NVNHKPSNTKVDKRVEPKSCDKTHTCPPCPAPEF |
| | EGGPSVFLFPPKPKDTLMISRTPEVTCVVVDVSH |
| | EDPEVKFNWYVDGVEVHNAKTKPREEQYNSTYRV |
| | VSVLTVLHQDWLNGKEYKCKVSNKALPASIEKTI |
| | SKAKGQPREPQVYTLPPSREEMTKNQVSLTCLVK |
| | GFYPSDIAVEWESNGQPENNYKTTPPVLDSDGSF |
| | FLYSKLTVDKSRWQQGNVFSCSVMHEALHNHYTQ |
| | KSLSLSPGK |
| VL: | EIVLTQSPGTLSLSPGERATLSCRASQRVSSSYL |
| SEQ ID NO:_10 | AWYQQKPGQAPRLLIYDASSRATGIPDRFSGSGS |
| | GTDFTLTISRLEPEDFAVYYCQQYGSLPWTFGQG |
| | TKVEIKRTVAAPSVFIFPPSDEQLKSGTASVVCL |
| | LNNFYPREAKVQWKVDNALQSGNSQESVTEQDSK |
| | DSTYSLSSTLTLSKADYEKHKVYACEVTHQGLSS |
| | PVTKSFNRGEC |

In a particular embodiment, the immune checkpoint inhibitor consist in the CTLA-4 blocking antibody (Ipilimumab) comprising:
   a heavy chain having a sequence set forth as SEQ ID NO:_11
   a light chain having a sequence set forth as SEQ ID NO:_12

The sequences of Ipilimumab antibody are indicated in the following Table 6:

| Mab | |
|---|---|
| Ipilimumab | |
| domains | Sequence |
| VH: | QVQLVESGGGVVQPGRSLRLSCAASGFTFSSYTM |
| SEQ ID NO:_11 | HWVRQAPGKGLEWVTFISYDGNNKYYADSVKGRF |
| | TISRDNSKNTLYLQMNSLRAEDTAIYYCARTGWL |
| | GPFDYWGQGTLVTVSSASTKGPSVFPLAPSSKST |
| | SGGTAALGCLVKDYFPEPVTVSWNSGALTSGVHT |
| | FPAVLQSSGLYSLSSVVTVPSSSLGTQTYICNVN |
| | HKPSNTKVDKRVEPKSCDKTHTCPPCPAPELLGG |
| | PSVFLFPPKPKDTLMISRTPEVTCVVVDVSHEDP |
| | EVKFNWYVDGVEVHNAKTKPREEQYNSTYRVVSV |
| | LTVLHQDWLNGKEYKCKVSNKALPAPIEKTISKA |
| | KGQPREPQVYTLPPSRDELTKNQVSLTCLVKGFY |
| | PSDIAVEWESNGQPENNYKTTPPVLDSDGSFFLY |
| | SKLTVDKSRWQQGNVFSCSVMHEALHNHYTQKSL |
| | SLSPGK |
| VL: | EIVLTQSPGTLSLSPGERATLSCRASQSVGSSYL |
| SEQ ID NO:_12 | AWYQQKPGQAPRLLIYGAFSRATGIPDRFSGSGS |
| | GTDFTLTISRLEPEDFAVYYCQQYGSSPWTFGQG |
| | TKVEIKRTVAAPSVFIFPPSDEQLKSGTASVVCL |
| | LNNFYPREAKVQWKVDNALQSGNSQESVTEQDSK |
| | DSTYSLSSTLTLSKADYEKHKVYACEVTHQGLSS |
| | PVTKSFNRGEC |

A further aspect of the invention relates to a method for treating solid tumors, comprising administering a subject in need thereof with amounts of an immune checkpoint inhibitor compound and a βig-h3 antagonist compound.

As used herein, the term “subject” denotes a human affected by a solid tumor.

The terms “cancer” and “tumors” refer to or describe the pathological condition in mammals that is typically characterized by unregulated cell growth. More precisely, in the use of the invention, diseases, namely tumors that express/secrete βig-h3 are most likely to respond to the βig-h3 antagonist after the restoration of CD8+ T cell activation. In particular, the cancer is associated with a solid tumor. Examples of cancers that are associated with solid tumor formation include breast cancer, uterine/cervical cancer, oesophageal cancer, pancreatic cancer, colon cancer, colorectal cancer, kidney cancer, ovarian cancer, prostate cancer, head and neck cancer, non-small cell lung cancer stomach cancer, tumors of mesenchymal origin (i.e; fibrosarcoma and rhabdomyoscarcoma) tumors of the central and peripheral nervous system (i.e; including astrocytoma, neuroblastoma, glioma, glioblatoma) thyroid cancer.

Preferably the solid tumor is selected from the group consisting of pancreatic cancer eosophage squamous cell carcinoma (Ozawa et al, 2014), gastric and hepatic carcinoma (Han et al, 2015), colon cancer (Ma et al, 2008), melanoma (Lauden et al, 2014).

In a preferred embodiment the solid tumor is a pancreatic cancer.

More preferably the pancreatic cancer is pancreatic ductal adenocarcinoma.

The terms “anti-tumoral CD8+ T cell response” means the natural ability of the CD8+ T cell to lyse cancer cells (Robbins and Kawakami, 1996, Romero, 1996)

Antibody

In another embodiment, the βig-h3 antagonist is an antibody (the term including antibody fragment or portion) that can block the interaction of βig-h3 with αVβ3 integrin.

In preferred embodiment, the βig-h3 antagonist may consist in an antibody directed against the βig-h3, in such a way that said antibody impairs the binding of a βig-h3 to αVβ3 integrin (“neutralizing antibody”).

Then, for this invention, neutralizing antibody of βig-h3 are selected as above described for their capacity to (i) bind to βig-h3 and/or (ii) reducing stiffening of TME and/or (iii) blocking the inhibiting CD8+ T cell activation.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of βig-h3. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant βig-h3 may be provided by expression with recombinant cell lines. Recombinant form of βig-h3 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Example of neutralizing anti-βig-h3 antibody is disclosed, for example, in Bae J S et al Acta Physiol 2014, 212, 306-315. The skilled artisan can use routine technologies to use the antigen-binding sequences of these antibodies (e.g., the CDRs) and generate humanized antibodies for treatment of PDAC as disclosed herein.

The inventors have cloned and sequenced the variable domain (VL) of the light chain, and the variable domain (VH) of the heavy chain of the monoclonal antibody 18B3. The location of the sequences encoding the complementarity determining regions (CDRs) of said antibody have been determined according to the IMGT numbering system. The IMGT unique numbering has been defined to compare the variable domains whatever the antigen receptor, the chain type, or the species (Lefranc M.-P., Immunology Today, 18, 509 (1997); Lefranc M.-P., The Immunologist, 7, 132-136 (1999).; Lefranc, Dev. Comp. Immunol., 27, 55-77 (2003).).

In a particular embodiment, the βig-h3 antagonist consist in the neutralizing anti-Pig-h3 antibody (18B3 antibody) comprising:
a heavy chain having a sequence set forth as SEQ ID NO:_13
a light chain having a sequence set forth as SEQ ID NO:_14

Therefore in a particular embodiment, the anti-βig-h3 antibody is an antibody comprising:

(a) a heavy chain wherein the variable domain comprises:
a H-CDR1 having a sequence set forth as SEQ ID NO:_15;
a H-CDR2 having a sequence set forth as SEQ ID NO:_16;
a H-CDR3 having a sequence set forth as SEQ ID NO:_17;

(b) a light chain wherein the variable domain comprises:
a L-CDR1 having a sequence set forth as SEQ ID NO:_18;
a L-CDR2 having a sequence set forth as SEQ ID NO:_19;
a L-CDR3 having a sequence set forth as SEQ ID NO:_20

The sequences of 18B3 antibody are indicated in the following Table 7:

| Mab 18B3 | |
|---|---|
| domains | Sequence |
| VH: | EVQLVESGGGLVKPGGSLKLSCAASGF |
| FR1-CDR1-FR2-CDR2- | TFSDYYMYWVRQTPEKRLEWVATISDG |
| FR3-CDR3-FR4 | GIYTYYPDSVKGRFTISRDSAKNNLYL |
| (SEQ ID NO:_13) | QMTSLKSDDTAMYYCVRGWDRYDSWFA |
| | CWGQGTLVTVSA |
| VL: | DIVMSQSPSSLVVSAGEKVTMTCKSSQ |
| FR1-CDR1-FR2-CDR2- | SLLYSSNQKNYLAWYRQKPGQSPKLLI |
| FR3-CDR3-FR4 | YWASTRESGVPDRFTGSGSGTDFTLTI |
| (SEQ ID NO:_14) | SSVKAEDLAVYYCQQYYRYPYTFGGGT |
| | KLEIK |
| VH-CDR1 | GFTFSDYY |
| (SEQ ID NO:_15) | |
| VH-CDR2 | ISDGGIYT |
| (SEQ ID NO:_16) | |
| VH-CDR3 | VRGWDRYDSWFAC |
| (SEQ ID NO:_17) | |
| VL-CDR1 | QSLLYSSNQKNY |
| (SEQ ID NO:_18) | |
| VL-CDR2 | WAS |
| (SEQ ID NO:_19) | |
| VL-CDR3 | QQYYRYPYT |
| (SEQ ID NO:_20) | |

In a particular embodiment, the βig-h3 antagonist consist in a neutralizing antibody that competes for binding to βig-h3 with the neutralizing anti-βig-h3 antibody (18B3 antibody).

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, N.J.) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard βig-h3 binding assays. The ability of a test antibody to inhibit the binding of antibodies of the present invention to βig-h3 demonstrates that the test antibody can compete with that antibody for binding to βig-h3; such an antibody may, according to non-limiting theory, bind to the same or a related (e.g., a structurally similar or spatially proximal) epitope on βig-h3 as the antibody with which it competes. Thus, another aspect of the invention provides antibodies that bind to the same antigen as, and compete with, the antibodies disclosed herein ((18B3 antibody). As used herein, an antibody “competes” for binding when the competing antibody inhibits βig-h3 binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of βig-h3. In some embodiments, the epitopes to which the present antibodies or antigen binding fragments bind are linear epitopes. In other embodiments, the epitopes to which the present antibodies or antigen binding fragments bind are non-linear, conformational epitopes.

The antibodies of the invention may be assayed for specific binding by any method known in the art. Many different competitive binding assay format(s) can be used for epitope binding. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York).

Aptamer

In another embodiment, the βig-h3 antagonist is an aptamer directed against βig-h3. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of βig-h3 are selected as above described for their capacity to (i) bind to βig-h3 and/or (ii) inhibit tumor cell growth and/or (iii) blocking the inhibiting CD8+ T cell activation.

Inhibitor of βIg-h3 Gene Expression

In still another embodiment, the βig-h3 antagonist is an inhibitor of βig-h3 gene expression. An “inhibitor of expression” refers to a natural or synthetic compound that has a biological effect to inhibit the expression of a gene. Therefore, an “inhibitor of βig-h3 gene expression” denotes a natural or synthetic compound that has a biological effect to inhibit the expression of βig-h3 gene.

In a preferred embodiment of the invention, said inhibitor of βig-h3 gene expression is a siRNA, an antisense oligonucleotide, a nuclease or a ribozyme.

Inhibitors of βig-h3 gene expression for use in the present invention may be based on antisense oligonucleotide constructs. Anti-sense oligonucleotides, including anti-sense RNA molecules and anti-sense DNA molecules, would act to directly block the translation of βig-h3 mRNA by binding thereto and thus preventing protein translation or increasing mRNA degradation, thus decreasing the level of βig-h3, and thus activity, in a cell. For example, antisense oligonucleotides of at least about 15 bases and complementary to unique regions of the mRNA transcript sequence encoding βig-h3 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of βig-h3 gene expression for use in the present invention. βig-h3 gene expression can be reduced by using small double stranded RNA (dsRNA), or a vector or construct causing the production of a small double stranded RNA, such that βig-h3 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Examples of said siRNAs against βig-h3 include, but are not limited to, those described in Chaoyu Ma (2008) Genes & Development 22:308-321.

Ribozymes can also function as inhibitors of βig-h3 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of βig-h3 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of βig-h3 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing βig-h3. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

As used herein, the term “active ingredients of the invention” is intended to refer to the βig-h3 antagonist compound and the immune checkpoint inhibitor compound as defined above.

The active ingredients of the invention may be administered in the form of a pharmaceutical composition, as defined below.

Preferably, the active ingredients of the invention are administered in a therapeutically effective amount.

By a “therapeutically effective amount” is meant a sufficient amount of the active ingredients of the invention to treat a solid tumor at a reasonable benefit/risk ratio applicable to any medical treatment.

In a preferred embodiment, the active ingredients of the invention are preferably administered by the intravenous route.

According to the invention, the active ingredients of the invention may be administered as a combined preparation for simultaneous, separate or sequential use in the treatment of solid tumor.

Since association of immune checkpoint inhibitors and βig-h3 antagonists had a synergistic effect on pancreatic cancer cells, the immune checkpoint inhibitors drug can advantageously be used at lower doses than in a treatment regimen wherein it is administered alone.

Therefore, in a preferred embodiment of the combination according to the invention, the immune checkpoint inhibitor drug is for use at a low dose, i.e. at a lower dose than the dose recommended when said drug is administered without said βig-h3 antagonist.

The skilled in the art can immediately determine a low dose for a given βig-h3 antagonist drug. Such a low dose notably depends on the cancer to be treated and on the therapeutic protocol.

In the frame of the present invention, by “low dose” is meant a dose that is inferior to the recommended dose that would be given to the patient when the immune checkpoint inhibitor is administered in the absence of the βig-h3 antagonist. Said low dose is preferably inferior by at least 10%, 15%, 20%, 25%, 50% or 75% to the recommended dose when combined to the usual therapeutic dose of immune checkpoint inhibitor.

The recommended dose that would be given to the patient when the immune checkpoint inhibitor is administered in the absence of the βig-h3 antagonist is known to the skilled in the art. Such a recommended dose can, for example, be found in the information provided by the authorities delivering marketing authorizations (e.g. in the EPARs published by the EMEA).

In a preferred embodiment, the βig-h3 antagonist of the invention is preferably administered by the intravenous route, the immune checkpoint inhibitor of the invention is preferably administered by the oral route.

Pharmaceutical Compositions According to the Invention

The present invention also provides a pharmaceutical composition comprising:

i. a βig-h3 antagonist (as defined here above),

ii. an immune checkpoint inhibitor (as defined here above); and

iii. a pharmaceutically acceptable carrier.

Pharmaceutical compositions formulated in a manner suitable for administration to humans are known to the skilled in the art. The pharmaceutical composition of the invention may further comprise stabilizers, buffers, etc.

The compositions of the present invention may, for example, be formulated and used as tablets, capsules or elixirs for oral administration, suppositories for rectal administration, sterile solutions or suspensions for administration by injection.

The choice of the formulation ultimately depends on the intended way of administration, such as e.g. an intravenous, intraperitoneal, subcutaneous or oral way of administration, or a local administration via tumor injection.

The pharmaceutical composition according to the invention may be a solution or suspension, e.g. an injectable solution or suspension. It may for example be packaged in dosage unit form.

In a preferred embodiment, the βig-h3 antagonist and the immune checkpoint inhibitor of the invention is preferably administered by the intravenous route.

The present invention also provides a pharmaceutical composition comprising:
i. a βig-h3 antagonist (as defined here above),
ii. an immune checkpoint inhibitor (as defined here above); and
iii. a pharmaceutically acceptable carrier. for use in the prevention or the treatment of solid tumor in a patient in need thereof.

In preferred embodiment, the solid tumor is selected from the list consisting of breast cancer, uterine/cervical cancer, oesophageal cancer, pancreatic cancer, colon cancer, colorectal cancer, kidney cancer, ovarian cancer, prostate cancer, head and neck cancer, non-small cell lung cancer stomach cancer, tumors of mesenchymal origin (i.e; fibrosarcoma and rhabdomyoscarcoma) tumors of the central and peripheral nervous system (i.e; including astrocytoma, neuroblastoma, glioma, glioblatoma) thyroid cancer.

Preferably the solid tumor is selected from the group consisting of pancreatic cancer eosophage squamous cell carcinoma, gastric and hepatic carcinoma, colon cancer, melanoma.

In a preferred embodiment the solid tumor is a pancreatic cancer.

More preferably the pancreatic cancer is pancreatic ductal adenocarcinoma.

The invention will be further illustrated by the following figures and examples. However, these examples and figures should not be interpreted in any way as limiting the scope of the present invention.

### FIGURES

FIG. 1: The impact of inducing the in vivo depletion of βig-h3 in KIC mice. (a) Experimental protocol used to induce antibody depletion. (b) Tumoral weights were quantified at the end of the experiment. (c) Impact of the combination anti-βig-h3 and anti-PD-1 Abs. The experiment was performed using 5-6 mice per group. (d) Quantification of the GrzB staining per tumoral area (on whole scan section). (e) Survival curves of untreated and anti-βig-h3 treated mice (f) survival curves of untreated and anti-βig-h3 and anti-PD-1 Abs treated mice. The median survival are shown in the tables. ns non significant, *P<0.05, **P<0.01, ****P<0.0001.

FIG. 2: βig-h3 depletion in established PDA leads to reduced tumor volume. (a) Experimental protocol used for antibody depletion. (b) Tumoral volume was quantified using ultrasound (Vevo2100®) in Ab-treated animals. (c) Representative immunohistochemistry for CK19 and cleaved caspase-3 in big-h3-treated (AB) and untreated (UT) KPC mice. Scale bar, 50 μm. (d) Quantification of PDA and PANIN areas based on CK19 staining and (e) Quantification of the results of staining for cleaved caspase-3. The experiment was performed using 5-6 mice per group. *P<0.05 and ***P<0.001

FIG. 3: βig-h3 depletion in established PDA reprograms tumor microenvironment in primary lesion and metastasis. (a) Experimental protocol used for antibody depletion. (b) Tumoral volume was quantified using ultrasound (Vevo2100®) in Ab-treated animals and represented in % to day 0. (c) Elastic Modulus quantification by AFM coupled with IF (based on CK19 and aSMA staining) in UT and AB treated KIC mice (3 independent mice per group, 100 force curves were measured per interest zone). (d) Quantification of total collagen (transmitted light) and thick fibers (polarized light) content. *P<0.05, ****P<0.0001.

FIG. 4: βig-h3 is expressed mainly in the stromal compartment. (a) Schematic representation of the isolated cell populations. (b) qPCR analysis of bigh3 levels in freshly isolated CAF and ductal cells. TATA-binding protein (TBP) was used as a control housekeeping gene. Relative expression levels were calculated using the equation 2^{-CT} Target/2^{-CT TBP}. The results shown are representative of 2 independent experiments that included 3 mice per group. (c) CAF or ductal cells were plated in complete medium or stimulated with 20 ng/ml of TGF-b1 for 48 h. The levels of secreted big-h3 were quantified using ELISA in the culture supernatants. The results shown are representative of 2 independent experiments that included 3 different CAF preparations and 2 different ductal preparations. *P<0.05; **P<0.01 and ***P<0.001

### EXAMPLES

The following examples describe some of the preferred modes of making and practicing the present invention. However, it should be understood that the examples are for illustrative purposes only and are not meant to limit the scope of the invention.

Material and Methods

Mice

The p48-Cre;Kras^{G12D} (KC), pdx1-Cre;Kras^{G12D};Ink4alArf^{fl/fl} (KIC) and pdx1-Cre;Kras^{G12D};p53^{R172H} (KPC) mice have been previously described²⁶⁻²⁸. All animal protocols were reviewed and approved in accordance with the guidelines provided by the Cancer Research Center Lyon Animal Care and Use Committee.

Collection of Tissue Samples from Mice

Normal and tumoral pancreas were washed in PBS, minced into small fragments and then incubated in collagenase solution (1 mg/ml collagenase V obtained from Roche in HBSS) at 37° C. for 20 min. The spleen and peripancreatic lymph nodes were homogenized and passed through a 70 μm cell strainer to achieve single cell suspensions. Red blood cells were lysed using NH4Cl lysis buffer.

Antibodies

For the in vivo studies, the following endotoxin-free antibodies were used: anti-CD8 (BioXcell; 2.43), anti-βigh3 18B3²⁹, anti-PD-1 and control polyclonal mouse Ig (BioXcell),

Isolation of Pancreas Cell Populations

Ductal cells and CAFs were isolated using anti-CD45, anti-PDGFR-PE and anti-EPCAM or CD45 antibodies and FACS sorting.

PDGFRα-PE isolated CAF (obtained from 3 different KC mice) were cultured and amplified in vitro. CAF or ductal cells were seeded at 10⁴ cells/well and then stimulated using mouse TGF-β1 at a final concentration of 20 ng/ml for 48 h. The CAF supernatants (CAF SNs) were then collected and used in the T cell suppression assays.

Functional T Cell Suppression Assay

Purified CD8+ T cells were labeled with 1 μM 5,6-carboxyfluorescein diacetate succinimidyl ester (CFSE, Invitrogen) at 37° C. for 20 min in serum-free RPMI. OT1 CFSE-labelled splenocytes were stimulated with OVA (SIINFEKL) peptide for 5 days in the presence or absence of recombinant human βig-h3 (rβig-h3) at a final concentration of 5 μg/ml. The antigen-specific suppression of CD8+ T cells was evaluated in co-culture assays in which splenocytes obtained from OT-1 transgenic mice (antigen-specific assays) were seeded in triplicate in 96-well round bottom plates (5×10⁵ cells/well). The splenocytes were cultured in the presence of CAF SN that was treated with or without anti-βig-h3 Ab and then stimulated with a cognate antigen, the OVA-derived peptide SIINFEKL (1 mg/ml; New England Peptide) for 3 days. Alternatively, mitomycin-treated-KC cells were co-cultured with CFSE-labelled pancreatic lymph node cells in the presence of a neutralizing anti-βigh3 Ab or control Ab (BioXCell, USA) at a final concentration of 6 μg/ml for 5 days. Proliferation was evaluated at the end of the culture period using flow cytometry for CFSE dilution.

Treatment of KPC and KIC Mice

KPC or KIC mice were treated twice a week for a period of 21 day and the sacrificed. Tumor volume monitoring was done by VevoScan in KPC mice. βigh3 was used at 8 micrograms/mouse and anti-PD-1 20 micrograms/mouse. For combo the injections were done separately in ip at the same time (twice a week).

Immunohistochemistry and Immunofluorescence

Slides with 4 μm-thick sections of mouse or human pancreatic tissues embedded in paraffin were deparaffinized. The sections were unmasked using unmasking solution (Vector H 3300), saturated with antibody diluent (Dako) for 30 minutes and then incubated with primary antibodies (anti-βig-h3, Sigma; anti-caspase-3, Cell Signaling; and CK19 Troma III, DSHB) that were diluted in antibody diluent overnight at 4° C. The sections were washed and then incubated with goat anti-rat biotinylated secondary antibodies (BD Biosciences; 1:200) for 1 h at RT. The remaining steps were performed using Vectastain ABC kits (Vector Labs). The slides were counterstained with hematoxylin.

Reverse Transcription and qPCR

RNA was extracted using a Qiagen kit from pelleted islets according to the manufacturer's instructions. RNA concentrations were measured using a Nanodrop spectrophotometer. Reverse transcription (RT) was assessed using equivalent quantities of extracted RNAs (superior to 300 ng). cDNA was used to perform quantitative polymerase chain reaction (qPCR) analyses with Power SYBR® Master Mix (Life Technologies). The following primers were used: TBP Forward 5′-TGGTGTGCACAGGAGCCAAG-3′(SEQ ID No 21) TBP Reverse 5′-TTCACATCACAGCTCCCCAC (SEQ ID No 22), and βig-h3 All-in-One™ qPCR (MQP028379) primers, which were obtained from GeneCopoeia.

Atomic Force Microscopy

We used AFM coupled with confocal microscopy to determine sequentially mechanical properties and pancreatic tissue domain identity. In AFM, the tip of a cantilever is pushed against the sample and the deflection of this cantilever is monitored. Using the stiffness constant of the lever, the deflection indicates the resisting force of the sample. Our protocol³⁰ allows us to measure the stiffness of sample very locally in a minimally invasive manner, by deforming the sample down to a depth of 100 nm. In order to investigate the stiffness patterns and the different domains of the pancreatic exocrine compartment during PDA (stromal compartment and pancreatic tumor cells) at high resolution we used the QNM (quantitative nanomechanical mapping) and the force volume protocols (Bruker). In these protocols the AFM probe oscillate at low frequency while horizontally scanning the sample and a force curve in generated each time the probe made contact with the sample. The elastic modulus of sample, reflecting the stiffness, is then extract from each curve applying the Sneddon (Hertz) model, yielding two-dimensional stiffness maps, where each pixel represents one force curve.

Statistical Analysis

P values were calculated using Student's t-test, (GraphPad Prism) as indicated in the figure legends. *P<0.05; **P<0.01; ***P<0.001; and ****P<0.0001. For multiple comparisons one way Anova with Tukey post test was used.

Results

βIg-h3 Depletion Increased Immune-Mediated Tumor Clearance In Vivo

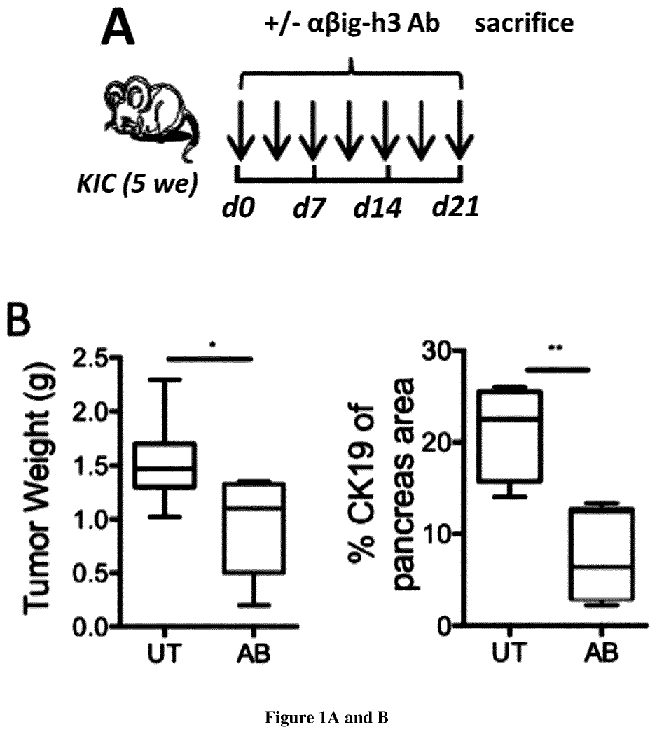

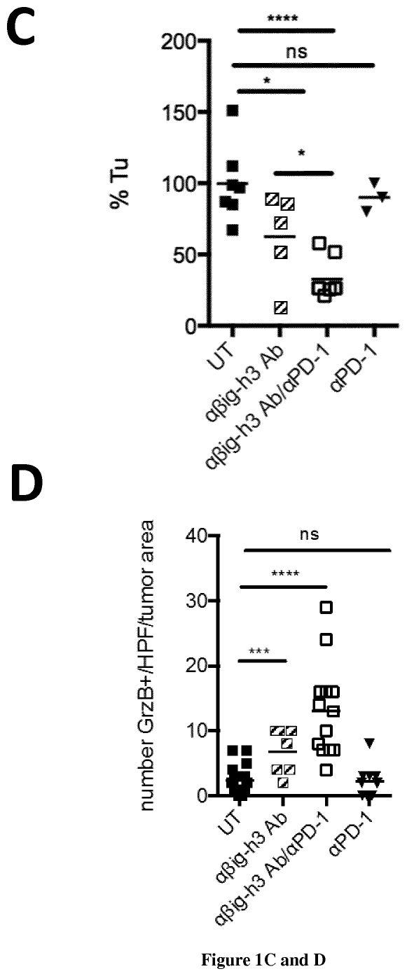

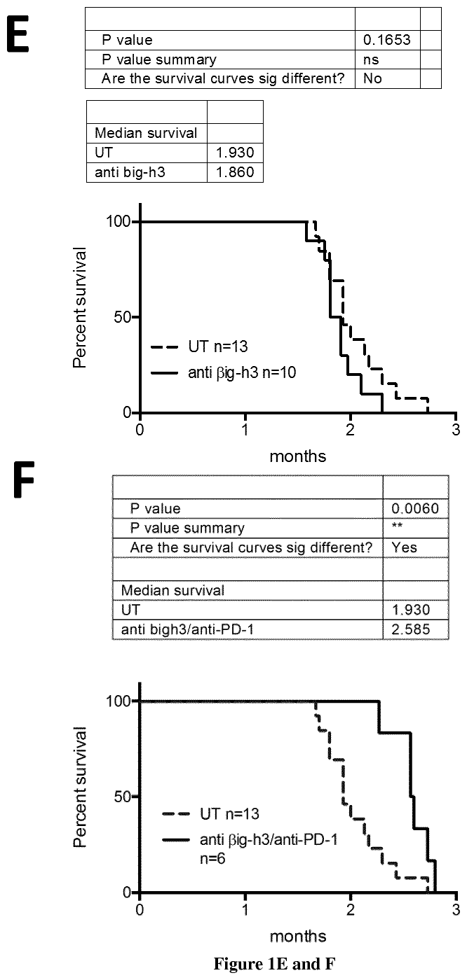

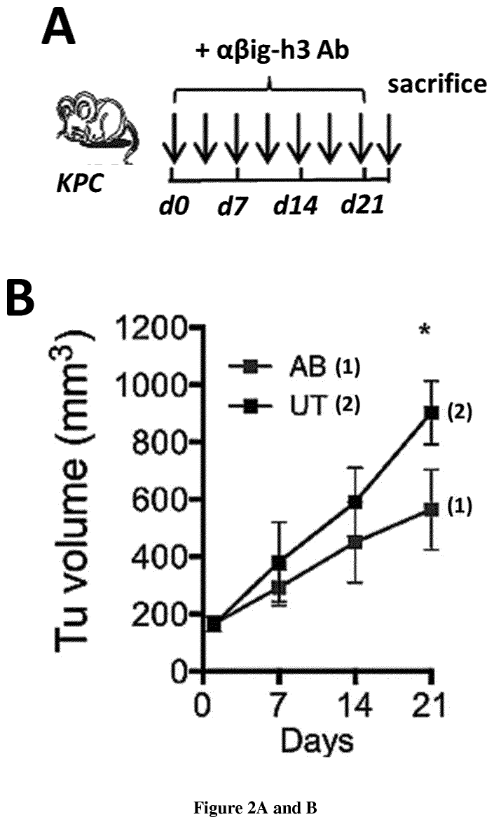

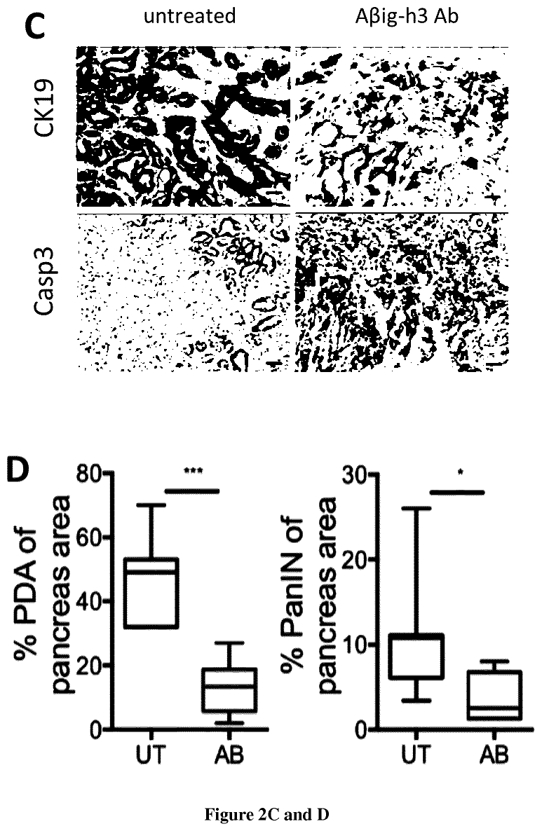

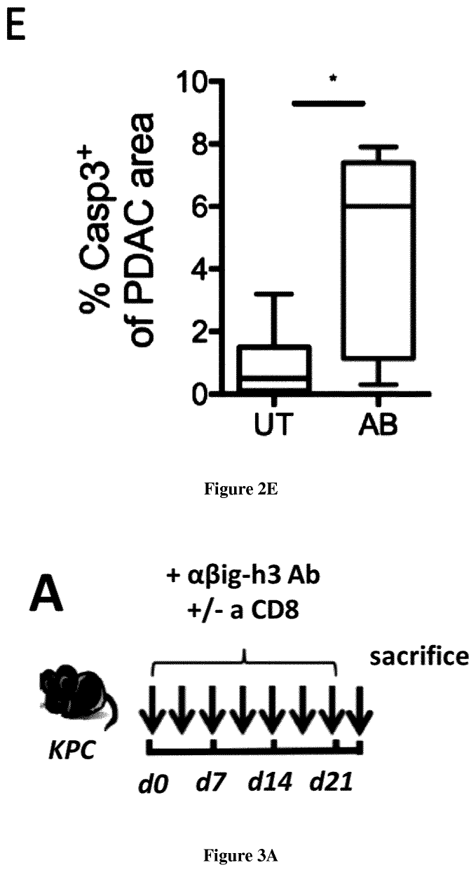

We evaluated the therapeutic potential of targeting βig-h3 in KPC and KIC mice, which are two well-established mouse models that develop aggressive pancreatic adenocarcinomas^{24, 28}. Whereas the KIC mice were injected twice a week with a βig-h3-depleting Ab for 21 days starting when the mice were 5 weeks old (FIG. 1A, B), the KPC mice were subjected to the same when the tumoral volume was between 100 and 200 mm³ (FIG. 2A, B). Interestingly, both the KPC and KIC mice that were injected with βig-h3-depleting antibodies had significantly smaller (approximately 38-40%) tumoral volumes than were observed in the untreated animals (FIG. 2B, 1B). The quantification of tumoral area, which was assessed using CK19 staining, revealed that there was a drastic reduction in tumoral area, from 46% to 13%, in the lesions within the pancreas of the βig-h3-depleting antibody-treated animals than in the untreated mice (FIG. 2C, D). Moreover, the PanIN area was also significantly smaller in the βig-h3-depleting antibody-treated animals than in the controls (FIG. 2C, D). The quantification of the number of cleaved-caspase-3⁺ cells showed that there were significantly more apoptotic cells in the βig-h3 Ab-treated mice than in the controls (FIG. 2E). More importantly, we detected an increase in the number of Granzyme B-positive cells that were in close contact with cleaved-caspase-3⁺ cells in the βig-h3 Ab-treated animals. Furthermore, in KIC mice the combination therapy (anti-βig-h3 and anti-PD-1 Abs) led to further synergistic effect and increased GrzB positive cells (FIG. 1C, D). Furthermore, the combination therapy (anti-βig-h3 and anti-PD-1 Abs) led to increased mouse survival (median survival 2.5 vs 1.9) whereas anti-βig-h3 treatment alone had no effect of the mouse survival (FIG. 1E, F).

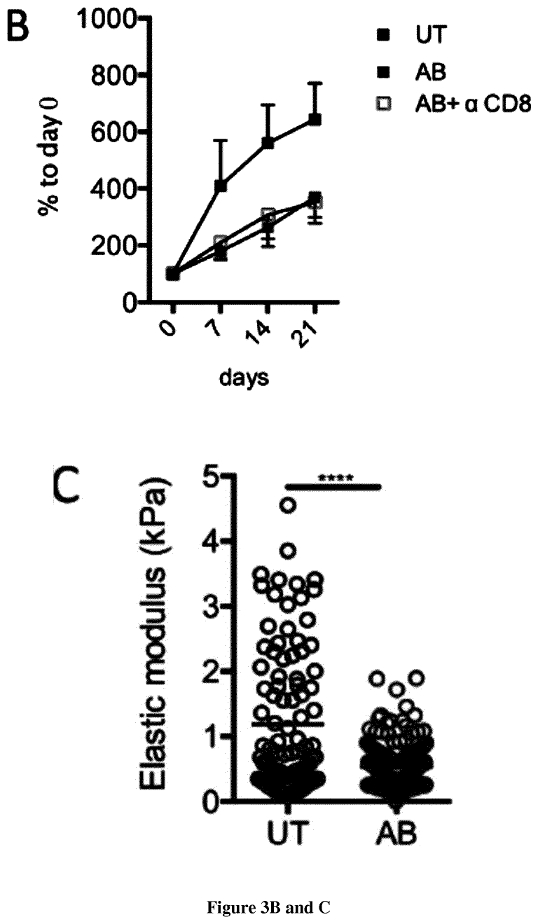

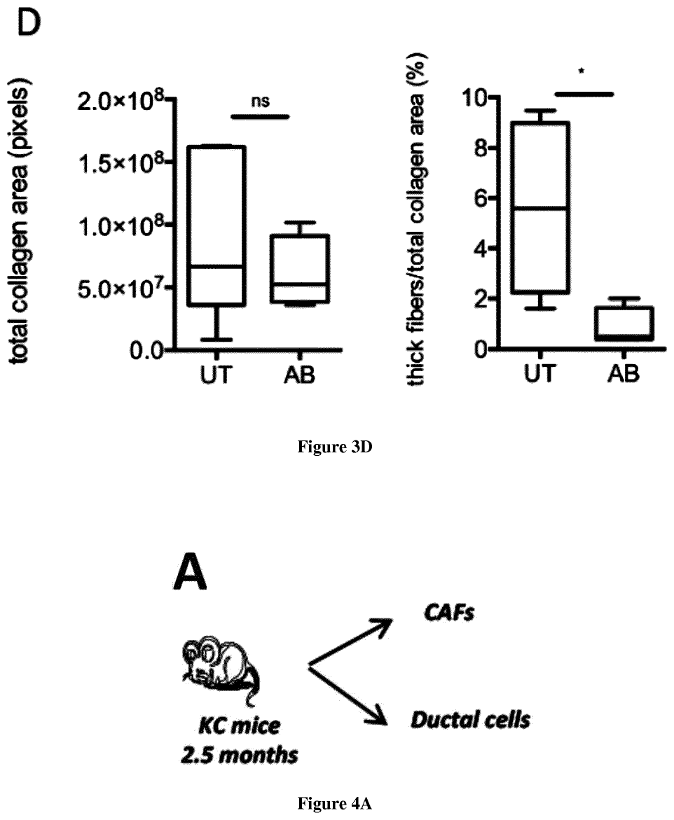

In order to find out if depletion of CD8+ T cells conjugated with anti-βig-h3 treatment in advanced lesions restored tumor growth, we performed co-injections in KPC mice (FIG. 3a, b). We found that CD8+ T cell depletion was not able to restore tumor growth in the context of βig-h3 neutralization. Since it was previously reported the βig-h3 binds to collagens, we checked by atomic force microscopy analysis the tissue rigidity and found that overall rigidity was reduced in anti-βig-h3 treated mice (FIG. 3c). These findings were corroborated with reduced collagen I thick fibers as determined in polarized light after Sirius Red staining, whereas the overall content of collagen was similar in untreated and Ab-treated animals (FIG. 3d). Furthermore, we recovered the liver UT or Ab injected KPC mice and found out that metastases were less numerous, smaller and more infiltrated by F4/80 cells in Ab treated animals. Altogether, these results strongly suggest that depletion of βig-h3 protein reprograms the tumor microenvironment at the primary lesion but also at a distant metastasis site in favor of an efficient anti-tumoral immune response.

βIg-h3 is Produced in the Stromal Compartment of Pancreatic Neoplastic and Tumor Lesions

Because βig-h3 was detected in pancreatic neoplastic and tumor lesions, we next investigated whether βig-h3 is produced by the tumor cells themselves or by the stroma-tumor microenvironment (TME). To resolve this issue, we performed co-immunofluorescence experiments using Cytokeratin19 (CK19), a marker of ductal tumor cells, and PDGRFα, which was previously shown to be a specific surface marker for CAFs (24). We found that βig-h3 expression was mainly localized in PDGRFα+ stromal cells. PDGFRα also co-localized with aSMA, another hallmark of myofibroblasts (25). These observations were further confirmed in the PDA from KIC mice. Interestingly, we found that βig-h3 expression was mutually exclusive with the expression of CK19 in all analyzed PanINs, suggesting that duct cells lack βig-h3 expression.

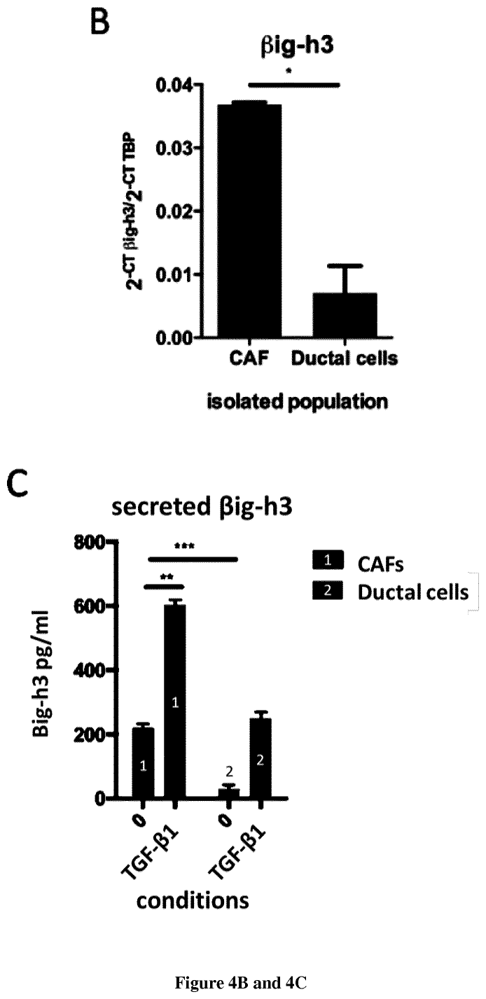

Next, we used CD45, EPCAM and PDGRFα, which are cell surface markers, to sort neoplastic duct cells (CD45-EPCAM+) and CAFs (CD45-PDGRFα+) in samples obtained from 2.5-month-old KC pancreatic tissues (FIG. 4a). We used EPCAM as a marker to sort live ductal cells since they co-expressed CK19 and EPCAM. Quantitative RT-PCR analysis was performed on the sorted cells, and the results confirmed that tgfβi was more strongly expressed in CAFs than in neoplastic ductal cells (FIG. 4b). To further validate this result, CAFs and ductal cells were cultured in vitro for 48 h in the presence or absence of TGF-β1 prior to quantification using a βig-h3 ELISA kit. An analysis of the cell culture supernatants confirmed that while CAFs produce βig-h3 ex vivo (219±12.3 pg/ml), it was barely detected in the supernatants of isolated ductal cells (28±13.5 pg/ml) (FIG. 4c). Interestingly, we found that stimulation with TGF-β1 potentiated the production of βig-h3 by both ductal cells and CAFs, yet the quantity of βig-h3 produced by TGF-β1-stimulated ductal cells never exceeded the basal level of βig-h3 that was produced by CAFs (FIG. 4c). Taken together, these data show that βig-h3 is produced mainly by PDGFRα+ CAFs within the stromal compartment of KC mice.

Discussion

The roles host immunity plays in regulating tumorigenesis and tumor progression are critical³¹. However, immune cells within the TME fail to exert an effective anti-tumor immune response³². This phenomenon is largely because an effective anti-tumoral immune response is unable to “reach” the tumoral zone and is maintained “physically and functionally” restricted to the surrounding microenvironment. In the TME, the stroma acts like a physical barrier that blocks access by both the immune system and chemotherapies to the tumor¹². While depleting the stroma in mice by blocking Hedgehog signaling has been shown to exert beneficial effects,³³ subsequent clinical trials that targeted stromal myofibroblasts in human PDA actually accelerated disease progression, which resulted in these clinical trials being halted. Therefore, the underlying mechanisms that allow the stroma to modulate the immune response have not been fully characterized. Here, we show that the stromal matrix protein βig-h3 directly restrains the anti-tumor immune response by inhibiting CD8+ T cell immunity in PDA. This strategy of immune evasion may therefore contribute to the resistance to immunotherapy that has been observed in this cancer.

PDA progression is associated with cellular and molecular changes in both the functional and stromal compartments of the pancreas. While lineage tracing experiments have shown that most preneoplastic lesions develop from pancreatic acinar cells via a process called Acinar to Ductal Metaplasia (ADM)³⁴, little is known about how the stroma is modulated and what its contributions are during the early stages of pancreatic cancer. Here, we show that βig-h3, a protein that was initially described as a secreted extracellular matrix protein that is produced mainly by fibroblasts, keratinocytes and muscle cells³⁵, is a novel protein that affects the pathophysiology of PDA. Our data provide insights into the role of βig-h3 in the modulation of the cellular interactions that occur in the TME during the early stages of PDA tumor development. While βig-h3 is not expressed in the exocrine compartment of the normal murine or human pancreas, we found that its expression is substantially increased within the stroma during the early stages of PDA. Interestingly, overexpressing βig-h3 in mice resulted in a higher incidence of spontaneous tumors than was observed in WT mice, whereas when βig-h3 was knocked out, the resulting mice were comparable to WT controls³⁶. These data suggest that targeting βig-h3 might have no substantial side effects. We found that βig-h3 was increased in patients with gastrointestinal cancers, including esophageal cancer, gastric cancer, hepatocarcinoma and PDA cancer³⁶. In patients with esophageal cancer, secreted βig-h3 was detected in the stroma using immunohistochemistry. Patients with high levels of βig-h3 in the stroma but not in tumor cells had a worse prognosis than those with low levels, indicating that this marker is a crucial contributor to a non-cell autonomous mechanism. Several lines of evidence indicate that βig-h3 densely accumulates in the stroma of PDA, where it exerts an immunosuppressive effect. First, we used T cell proliferation assays (using either a recombinant molecule or secreted in CAF supernatants) and found that βig-h3 exerted a suppressive effect by reducing antigen-specific activation and proliferation. Here, we provide the first evidence showing that the use of a depleting Ab against secreted βig-h3 restored tumor-specific CD8+ T cell proliferation and activation and reduced cell exhaustion, which was measured using PD-1 and Tim-3 expression in vitro. Furthermore, βig-h3 binds to and induces signals via integrin 133 (CD61), which is highly expressed on infiltrating CD8+ T cells and leads to the stabilization of Hic-5 that binds to Lck Y505 blunting the signal transduction. Moreover, the depletion of βig-h3 protein leads to the reprogramming of F4/80 macrophages that will produce cytotoxic molecules upon ingestion of the Ag/Ab complexes. Second, the depletion of βig-h3 protein in vivo using an Ab strategy was accompanied by an increase in the GrzB⁺ response. In case of rapid aggressive lesion development, the combination therapy with anti-PD-1 has a synergistic effect (KIC mice). Third, the immune-mediated elimination of subcutaneously injected tumor cells was fully rescued by CD8+ T cell depletion, indicating that the βig-h3 protein plays a central role in disrupting an effective anti-tumoral response during the early stages of neoplasia. More importantly, the relevance of this immune modulatory mechanism during more advanced stages of pancreatic cancer was further demonstrated when we depleted the protein in already established PDA and found out that the tumor microenvironment was reprogrammed not only at the primary tumor but also at the metastasis site raising the exciting possibility that targeting βig-h3 may bolster immune-mediated anti-tumor efficacy in patients.

### REFERENCES

Throughout this application, various references describe the state of the art to which this invention pertains. The disclosures of these references are hereby incorporated by reference into the present disclosure.
1. Kern S E, Shi C, Hruban R H. The complexity of pancreatic ductal cancers and multidimensional strategies for therapeutic targeting. J Pathol 2011; 223:295-306.
2. Hezel A F, Kimmelman A C, Stanger B Z, et al. Genetics and biology of pancreatic ductal adenocarcinoma. Genes & development 2006; 20:1218-49.
3. Apte M V, Haber P S, Applegate T L, et al. Periacinar stellate shaped cells in rat pancreas: identification, isolation, and culture. Gut 1998; 43:128-33.
4. Ene-Obong A, Clear A J, Watt J, et al. Activated pancreatic stellate cells sequester CD8+ T cells to reduce their infiltration of the juxtatumoral compartment of pancreatic ductal adenocarcinoma. Gastroenterology 2013; 145:1121-32.
5. Heinemann V, Reni M, Ychou M, et al. Tumour-stroma interactions in pancreatic ductal adenocarcinoma: rationale and current evidence for new therapeutic strategies. Cancer Treat Rev 2014; 40:118-28.
6. Zhang Y, Ertl H C. Depletion of FAP+ cells reduces immunosuppressive cells and improves metabolism and functions CD8+T cells within tumors. Oncotarget 2016; 7:23282-99.
7. Galon J, Costes A, Sanchez-Cabo F, et al. Type, density, and location of immune cells within human colorectal tumors predict clinical outcome. Science 2006; 313:1960-4.
8. Balachandran V P, Luksza M, Zhao J N, et al. Identification of unique neoantigen qualities in long-term survivors of pancreatic cancer. Nature 2017; 551:512-516.
9. De Monte L, Reni M, Tassi E, et al. Intratumor T helper type 2 cell infiltrate correlates with cancer-associated fibroblast thymic stromal lymphopoietin production and reduced survival in pancreatic cancer. J Exp Med 2011; 208:469-78.
10. Brahmer J R, Tykodi S S, Chow L Q, et al. Safety and activity of anti-PD-L1 antibody in patients with advanced cancer. N Engl J Med 2012; 366:2455-65.
11. Clark C E, Hingorani S R, Mick R, et al. Dynamics of the immune reaction to pancreatic cancer from inception to invasion. Cancer research 2007; 67:9518-27.
12. Provenzano P P, Cuevas C, Chang A E, et al. Enzymatic targeting of the stroma ablates physical barriers to treatment of pancreatic ductal adenocarcinoma. Cancer Cell 2012; 21:418-29.
13. Skonier J, Neubauer M, Madisen L, et al. cDNA cloning and sequence analysis of beta ig-h3, a novel gene induced in a human adenocarcinoma cell line after treatment with transforming growth factor-beta. DNA and cell biology 1992; 11:511-22.
14. Bae J S, Lee S H, Kim J E, et al. Betaig-h3 supports keratinocyte adhesion, migration, and proliferation through alpha3beta1 integrin. Biochem Biophys Res Commun 2002; 294:940-8.
15. Billings P C, Whitbeck J C, Adams C S, et al. The transforming growth factor-beta-inducible matrix protein (beta)ig-h3 interacts with fibronectin. J Biol Chem 2002; 277:28003-9.
16. Hashimoto K, Noshiro M, Ohno S, et al. Characterization of a cartilage-derived 66-kDa protein (RGD-CAP/beta ig-h3) that binds to collagen. Biochimica et biophysica acta 1997; 1355:303-14.
17. Nam J O, Kim J E, Jeong H W, et al. Identification of the alphavbeta3 integrin-interacting motif of betaig-h3 and its anti-angiogenic effect. J Biol Chem 2003; 278:25902-9.
18. Ohno S, Noshiro M, Makihira S, et al. RGD-CAP ((beta)ig-h3) enhances the spreading of chondrocytes and fibroblasts via integrin alpha(1)beta(1). Biochim Biophys Acta 1999; 1451:196-205.
19. Ma C, Rong Y, Radiloff D R, et al. Extracellular matrix protein betaig-h3/TGFBI promotes metastasis of colon cancer by enhancing cell extravasation. Genes Dev 2008; 22:308-21.
20. Patry M, Teinturier R, Goehrig D, et al. betaig-h3 Represses T-Cell Activation in Type 1 Diabetes. Diabetes 2015; 64:4212-9.
21. Turtoi A, Musmeci D, Wang Y, et al. Identification of novel accessible proteins bearing diagnostic and therapeutic potential in human pancreatic ductal adenocarcinoma. Journal of proteome research 2011; 10:4302-13.
22. Kaiser M F, Johnson D C, Wu P, et al. Global methylation analysis identifies prognostically important epigenetically inactivated tumor suppressor genes in multiple myeloma. Blood 2013; 122:219-26.
23. Kang S, Dong S M, Park N H. Frequent promoter hypermethylation of TGFBI in epithelial ovarian cancer. Gynecol Oncol 2010; 118:58-63.
24. Aguirre A J, Bardeesy N, Sinha M, et al. Activated Kras and Ink4a/Arf deficiency cooperate to produce metastatic pancreatic ductal adenocarcinoma. Genes & development 2003; 17:3112-26.
25. Hingorani S R, Petricoin E F, Maitra A, et al. Preinvasive and invasive ductal pancreatic cancer and its early detection in the mouse. Cancer Cell 2003; 4:437-50.
26. Aguirre A J, Bardeesy N, Sinha M, et al. Activated Kras and Ink4a/Arf deficiency cooperate to produce metastatic pancreatic ductal adenocarcinoma. Genes Dev 2003; 17:3112-26.
27. Ascher E, Markevich N, Schutzer R W, et al. Cerebral hyperperfusion syndrome after carotid endarterectomy: predictive factors and hemodynamic changes. J Vasc Surg 2003; 37:769-77.
28. Hingorani S R, Wang L, Multani A S, et al. Trp53R172H and KrasG12D cooperate to promote chromosomal instability and widely metastatic pancreatic ductal adenocarcinoma in mice. Cancer Cell 2005; 7:469-83.
29. Bae J S, Lee W, Son H N, et al. Anti-transforming growth factor beta-induced protein antibody ameliorates vascular barrier dysfunction and improves survival in sepsis. Acta Physiol (Oxf) 2014; 212:306-15.
30. Milani P, Mirabet V, Cellier C, et al. Matching Patterns of Gene Expression to Mechanical Stiffness at Cell Resolution through Quantitative Tandem Epifluorescence and Nanoindentation. Plant Physiol 2014; 165:1399-1408.
31. Grivennikov S I, Greten F R, Karin M. Immunity, inflammation, and cancer. Cell 2010; 140:883-99.
32. Ruffell B, DeNardo D G, Affara N I, et al. Lymphocytes in cancer development: polarization towards pro-tumor immunity. Cytokine Growth Factor Rev 2010; 21:3-10.
33. Olive K P, Jacobetz M A, Davidson C J, et al. Inhibition of Hedgehog signaling enhances delivery of chemotherapy in a mouse model of pancreatic cancer. Science 2009; 324:1457-61.
34. Strobel O, Dor Y, Alsina J, et al. In vivo lineage tracing defines the role of acinar-to-ductal transdifferentiation in inflammatory ductal metaplasia. Gastroenterology 2007; 133:1999-2009.
35. LeBaron R G, Bezverkov K I, Zimber M P, et al. Beta IG-H3, a novel secretory protein inducible by transforming growth factor-beta, is present in normal skin and promotes the adhesion and spreading of dermal fibroblasts in vitro. J Invest Dermatol 1995; 104:844-9.
36. Han B, Cai H, Chen Y, et al. The role of TGFBI (betaig-H3) in gastrointestinal tract tumorigenesis. Mol Cancer 2015; 14:64.

1. (canceled)

2. The method according to claim 16, wherein said immune checkpoint inhibitor is anti-PD-L1/PD-1 antibody.

3. The method according to claim 16, wherein said βig-h3 antagonist is an anti βig-h3 antibody.

4. The method according to claim 16, wherein said solid tumor is selected from the list consisting of pancreatic cancer eosophage squamous cell carcinoma, gastric and hepatic carcinoma, colon cancer, and melanoma.

5. The method according to claim 16, wherein said solid tumor is pancreatic cancer.

6. A method for enhancing sensitivity of a patient suffering from a solid tumor to an immune checkpoint inhibitor, comprising
administering to the patient a therapeutically effective amount of a βig-h3 antagonist.

7. The method according to claim 6 wherein said immune checkpoint inhibitor is anti-PD-L1/PD-1 antibody.

8. The method according to claim 6, wherein said βig-h3 antagonist is an anti βig-h3 antibody.

9. The method according to claim 6, wherein said solid tumor is selected from the list group consisting of pancreatic cancer, oesophagus squamous cell carcinoma, gastric and hepatic carcinoma, colon cancer, and melanoma.

10. The method according to claim 9, wherein said solid tumor is pancreatic cancer.

11. A pharmaceutical composition comprising:
i. a βig-h3 antagonist,
ii. a immune checkpoint inhibitor and
iii. a pharmaceutically acceptable carrier.

12. The pharmaceutical composition according to claim 11 wherein said immune checkpoint inhibitor is anti-PD-L1/PD-1 antibody.

13. The pharmaceutical composition according to claim 11 wherein said βig-h3 antagonist is an anti βig-h3 antibody.

14. A method of preventing or treating a solid tumor in a patient in need thereof, comprising
administering to the patient a therapeutically effective amount of the pharmaceutical composition of claim 11.

15. The method according to claim 14 wherein said solid tumor is selected from the group consisting of pancreatic cancer, oesophagus squamous cell carcinoma, gastric and hepatic carcinoma, colon cancer, and melanoma.

16. A method for treating solid tumors in a subject in need thereof, comprising administering to the subject a therapeutically effective amount of an immune checkpoint inhibitor and a βig-h3 antagonist.


SEQUENCE LISTING (22 sequences)

SEQ ID NO: 1 - protein, 447 aa, Artificial
  Synthetic VH Pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, Artificial
  Synthetic VL Pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 440 aa, Artificial
  Synthetic VH Nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 214 aa, Artificial
  Synthetic VL  Nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial
  Synthetic VH Atezolizumab
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial
  Synthetic VL Atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, Artificial
  Synthetic VH  Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial
  Synthetic VL Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - protein, 451 aa, Artificial
  Synthetic VH  Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial
  Synthetic VL Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial
  Synthetic VH Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial
  Synthetic VL Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 120 aa, Artificial
  Synthetic VH 18B3
EVQLVESGGGLVKPGGSLKLSCAASGFTFSDYYMYWVRQTPEKRLEWVATISDGGIYTYYPDSVKGRFTISRDSAKNNLYLQMTSLKSDDTAMYYCVRGWDRYDSWFACWGQGTLVTVSA

SEQ ID NO: 14 - protein, 113 aa, Artificial
  Synthetic VL 18B3
DIVMSQSPSSLVVSAGEKVTMTCKSSQSLLYSSNQKNYLAWYRQKPGQSPKLLIYWASTRESGVPDRFTGSGSGTDFTLTISSVKAEDLAVYYCQQYYRYPYTFGGGTKLEIK

SEQ ID NO: 15 - protein, 8 aa, Artificial
  Synthetic VH_CDR1 18B3
GFTFSDYY

SEQ ID NO: 16 - protein, 8 aa, Artificial
  Synthetic VH_CDR2 18B3
ISDGGIYT

SEQ ID NO: 17 - protein, 13 aa, Artificial
  Synthetic VH_CD3 18B3
VRGWDRYDSWFAC

SEQ ID NO: 18 - protein, 12 aa, Artificial
  Synthetic VL_CDR1 18B3
QSLLYSSNQKNY

SEQ ID NO: 19 - protein, 3 aa, Artificial
  Synthetic VL_CDR2 18B3
WAS

SEQ ID NO: 20 - protein, 9 aa, Artificial
  Synthetic VL_CDR3
QQYYRYPYT

SEQ ID NO: 21 - DNA, 20 bp, Artificial
  Synthetic TBP Forward  primer
tggtgtgcacaggagccaag

SEQ ID NO: 22 - DNA, 20 bp, Artificial
  Synthetic TBP Reverse primer
ttcacatcacagctccccac